(19) United States
(12) Patent Application Publication
(10) Pub. No.: US 2019/0240448 A1
(43) Pub. Date: Aug. 8, 2019

(54) DRAINAGE AND ANCHORING SYSTEM FOR AN INDWELLING URINARY CATHETER

(71) Applicant: Jonah Murdock, Chevy Chase, MD (US)

(72) Inventor: Jonah Murdock, Chevy Chase, MD (US)

(21) Appl. No.: 15/887,763

(22) Filed: Feb. 2, 2018

(51) Int. Cl. A61M 25/00; A61M 25/04

(52) U.S. Cl. CPC A61M 25/0017; A61M 25/1002; A61M 25/0067; A61M 25/04

(57) ABSTRACT

This invention relates to catheters, and more particularly to Foley, indwelling or self-retaining catheters. Disclosed are particular drainage port positions along the catheter and anchor shapes that facilitate complete drainage of a hollow organ or bladder in which the catheter is inserted. Methods described herein may prevent the pathological expansion of a balloon anchor member portion of an indwelling catheter from being deployed and expanded prior to advancing in the bladder and beyond the urethra.

### FIELD OF THE INVENTION

This invention relates to catheters, and more particularly to Foley, indwelling or self-retaining catheters.

### BACKGROUND

An indwelling urinary catheter (commonly referred to as a “Foley catheter”) is a flexible tube that is passed through the urethra and into the bladder to drain urine for a period of time. Foley, indwelling or retention catheters are constructed with an elastic inflatable balloon surrounding the hollow catheter shaft near the distal end. The balloon, in a deflated condition snugly engages the shaft so that it minimizes the impedance of the catheter shaft as it is passed through the urethra until its drainage eye or port reaches the desired position, e.g., well within the interior of the bladder, whereupon the balloon is inflated through a separate inflation lumen passing through the shaft; the inflation medium usually is water, normal saline, or glucose solution. The inflation lumen is sealed with the balloon in an inflated condition to prevent withdrawal of the catheter. The catheter can then be retracted such that the balloon contacts an inner surface of the bladder wall to confirm it is securely anchored or retained within bladder. One or more additional lumens may be carried within or on the catheter to deliver instruments, energy, optics, diagnostics, therapeutic agents, gases or liquids. In normal usage, such a catheter can remain in position for an extended period of time ranging from hours to as much as several weeks, during all of which time the retention balloon must remain inflated to ensure that the catheter is not accidentally withdrawn. At the time withdrawal is desired, the seal is released, the inherent elasticity of the balloon wall causing the balloon to collapse to its original uninflated condition snugly engaging the shaft with accompanying ejection of the inflation medium through its lumen.

Indwelling or Foley catheters are commonly used medical procedures wherein the patient is: anesthetized or sedated for surgery or other medical care, suffering from an enlarged prostate such that urine flow from the bladder is cut off, suffering from acute urinary retention, is unable due to paralysis or physical injury to use either standard toilet facilities or urinals; having urethral surgery, having a ureterectomy, suffering from kidney disease and whose urine output must be constantly and accurately measured, having a cesarean section, having a hysterectomy, suffering from a genital injury, anorexic an unable to use standard toilets due to physical weakness, suffering from fibromyalgia and cannot control their bladder.

However, there can be complications with using such catheters and catheter-associated urinary tract infection (UTI) is one of the most common type of hospital-acquired infections. As the kidneys empty into the bladder, a UTI can also result in infected kidneys. A IT can be caused by a bio film of bacteria traveling up the catheter and into the bladder or by stagnant urine within the bladder. Urine containing infectious bacteria may stagnate because of clogging of the distal drainage port or because of pooling around and below the periphery of the balloon.

Indwelling catheters are also used in various medical procedures and organs other than the bladder. For example, such catheters have been used in other procedures to drain, irrigate, deliver instruments, therapeutic or diagnostic agents involving the cervix, nasal passage, throat, or any other suitable hollow organ. As in the bladder similar complications and infections can occur in an organ that is inadequately drained of bodily fluids, such as blood, lymph, mucus, or puss.

The first two figures present views of typical indwelling catheters and basic physical anatomy with implanted catheters. FIG. 1 shows various anchored or self-retaining indwelling urinary catheters from the prior art; FIG. 2 shows a typical Foley catheter from the prior art inserted within the male and female anatomy. Not that some catheters utilize expandable anchors instead of balloons but they are prone to unintended expulsion under enough force and simply compress whereas balloon like anchors filled with liquid or gas tend to act incompressibly under most physiological conditions.

### SUMMARY OF THE INVENTION

In one aspect of the invention a catheter device is provided that includes an elongate primary tube having a proximal exit end for collecting or dispensing fluids and a distal receiving end. The primary tube can have one or more drainage apertures for receiving fluids. The catheter has an inflatable or expandable anchor carried proximal to the distal end of the primary tube which can carry a secondary tube for passing or receiving a substance to the inflatable or expandable anchor device. The anchor is configured such that once delivered within and beyond a urethra and into a bladder and subsequently inflated or expanded therein, the anchor will resist the expulsion of the distal end of the primary tube end out of the bladder upon flow of fluid within bladder or during micturition; and wherein the one or more drainage apertures are positioned below and proximate to said inflatable anchor device along said primary tube such that the fluids may pass into the primary tube.

The drainage apertures can be operable to be in partial fluid communication with the bladder outlet or inter urethral orifice when the catheter is inserted in the bladder and the inflatable anchor is inflated. In certain embodiments, the anchor can have an upper (facing away from the urethra) and lower surface (adjacent to the urethral opening) and can be at least partially concave or present a can cavity adjacent to one or more of the drainage apertures in the catheter. A second drainage aperture can be located at the distal end of the primary tube above the upper surface of the inflatable anchor or flush with its upper surface.

The drainage apertures can alternatively be positioned radially along said primary tube. They can also be oblong in shape and mounted along a long axis of the primary tube. Alternatively the drainage apertures be configured out of a circumferential mesh. The shape of the inflatable anchor may include a sphere, toroid, cylinder, or ellipsoid. It may also include radial surface indentations configured to channel fluid below the anchor towards the one or more drainage apertures. Alternatively it may include one or more surface protrusions configured to provide a gap between an outer surface of the anchor and bladder wall configured to channel fluid below the anchor towards the one or more drainage apertures.

In another embodiment, an anchorable irrigation device for releasing fluid from a hollow structure within a patient is provided. The device includes an elongate primary tube having a proximal exit end for collecting or dispensing fluids and a distal receiving end and one or more drainage apertures for receiving fluids. It also includes an inflatable or expandable anchor carried proximal to the distal end of the primary tube which and carry a secondary tube for passing or receiving a substance the anchor.

The anchor is configured such that once delivered within the interior of the hollow structure and subsequently inflated or caused or allowed to expand therein the anchor will resist the expulsion of the distal end of said primary tube end out of said structure upon flow of fluid within said structure. The one or more drainage apertures are laterally positioned below and proximate the anchor device along the primary tube such that said fluids may pass into the primary tube and out of the hollow structure. The anchor can present a concavity adjacent or proximal to one or more of the drainage apertures.

One or more aspects of methods of catheterization described herein involve preventing infection within a patient's bladder. The methods can include the steps of: inserting a catheter having an expandable anchor at its distal end and a drainage aperture positioned below said anchor through a urethra and into and beyond a bladder outlet into the bladder, causing or allowing the anchor to expand; expanding the anchor, positioning at least a portion of said aperture between the anchor and a lower portion of said bladder thereby fully evacuating the bladder and preventing urine from remaining therein and causing an infection. The method can also include the step of expanding the anchor via inflation.

In another method disclosed herein, a method of irrigating a hollow structure within a patient is disclosed. One or more aspects of the method can include the following steps: inserting a catheter having an expandable anchor at its distal end and a drainage aperture positioned below the anchor through the surface of the hollow structure and into and within the hollow structure; expanding the anchor; and positioning at least a portion of said aperture between said anchor and an interior surface of said hollow structure. The hollow structure can a bladder and the method could involve piecing a hole the bladder and causing or allowing the bladder wall to seal around it.

Another method described herein involves preventing the pathological expansion of an expandable anchor attached to the distal end of an indwelling catheter within a urethra and can included the following steps; inserting an indwelling catheter within a urethra, the catheter having a elongate primary tube having a proximal exit end for collecting or dispensing fluids and a distal receiving end, wherein the primary tube has one or more drainage apertures for receiving fluids, an inflatable anchor carried proximal to the distal end of said primary tube, said primary tube carrying a secondary tube for passing or receiving a substance to said inflatable anchor device and wherein the one or more drainage apertures are laterally positioned below and proximate to said inflatable anchor device along the primary tube such that said fluids may pass into the primary tube; continuing to insert catheter into the urethra until fluid appears or emerges from the proximal collecting end of said primary tube thereby indicating the inflatable member has been inserted beyond the urethra; and inflating the in inflatable anchor and securing the drainage port within the bladder.

### BRIEF DESCRIPTION OF DRAWINGS

FIG. 1 shows various anchored or self-retaining indwelling urinary catheters from the prior art.

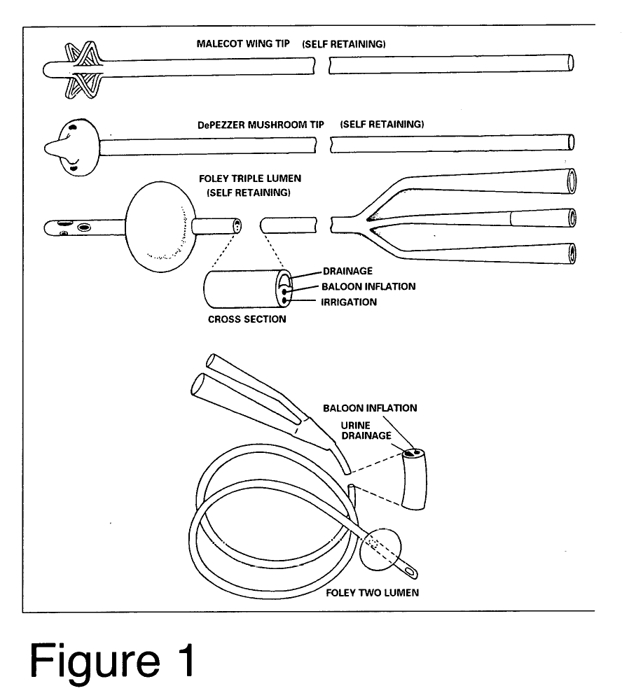

FIG. 2 shows a typical Foley catheter from the prior art inserted within the male and female anatomy.

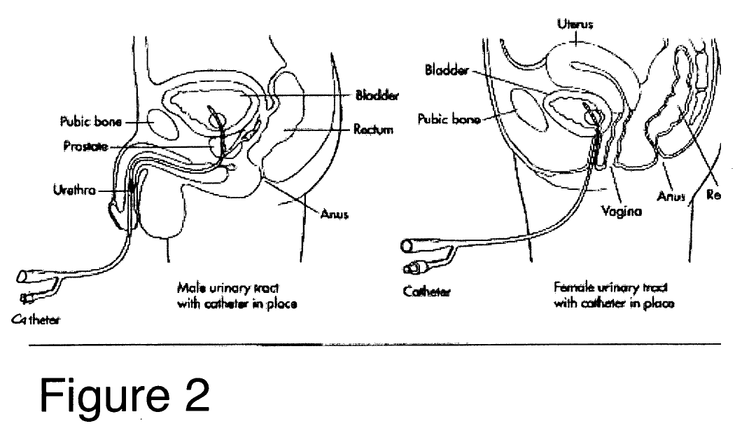

FIGS. 3A-C are cross-sectional side views of an embodiment of the invention inserted in a bladder and urethra with various port arrangements.

FIGS. 4A and 4B are cross-sectional side views of an embodiment of the invention having a balloon with an “umbrella” shaped configuration such that the balloon presents a concave surface on its lower portion.

FIGS. 5 A-d show perspective views of various embodiments of the invention wherein a catheter has expandable balloon members embodied as a toroid, disc, radial four-legged extensions and tripod extensions.

FIGS. 6A and 6B show a top view and a side view of an embodiment of the invention having a balloon or expandable member having exterior channels to allow the flow of liquid across its exterior.

FIGS. 7A-E show side views of a bladder and urethra wherein the port of a catheter device is positioned at various locations relative to the intersection of the bladder and urethra.

The following is a listing of corresponding elements and reference numbers as used in the various sections Drawings and Description though some elements and limitations may be shown and described herein but lack such reference numbers:
1 Bladder
2 Inner surface or wall of a hollow bladder or organ
3 Bladder orifice or outlet; internal bladder orifice
4 Junction of bladder orifice and inner surface of a hollow bladder or organ
5 Urethra
10 Catheter body
15 Secondary tube carried by the catheter for passing fluid or gas to the anchor or outside of the catheter
20 Balloon, expandable anchor or retaining device
21 Under/lower surface of a balloon, expandable anchor or retaining device presenting a concavity
22 Channels, indentations, ridges, concavities on or along balloon surface
23 Dimples or protrusions on the surface of the balloon
30 Distal insertion tip of catheter
40 Drainage port, opening or slit for receiving fluid positioned below a balloon, expandable anchor or retaining device
41 Drainage port, opening or slit positioned above a balloon, expandable anchor or retaining device
50 Hollow interior of catheter body
60 Proximal end of catheter body
61 Collection port or opening for receiving fluid from a distal drainage port
62 Delivery port for delivering or receiving media to the balloon, expandable anchor or retaining device
63 Auxiliary port for irrigation or delivering instrumentation, therapeutics, or diagnostics within the bladder, urethra or organ through an opening along the catheter
100 Anchorable indwelling catheter according to various embodiments describe herein

### DESCRIPTION

Various improvements to catheters have been made to reduce the physical irritation and complications inflicted on the bladder, urethra and other organs by changing the shape, size, material, and coatings of the balloon and catheter body. Presented herein are improved methods and devices involving new types of urinary bladder drainage catheters. It should be noted that Dr. Foley invented the Foley catheter more than seventy years ago, and, as noted above, use of it does involve complications. These complications may arise from its unique design. It has a spherical balloon which inflates to keep the Foley catheter in the bladder. The drainage port for urine is positioned at the top end of the catheter, just beyond the balloon.

In contrast, one or more catheter embodiments according to this disclosure employ an expandable balloon or buoyancy device at its end with the drainage port just proximal to the lower portion of the balloon. The balloon can include a variety of embodiments having different shapes and features can contribute to the primary goal of effective drainage of the bladder and maintaining its position. The material of the balloon can be flexible, elastic or inelastic. It can be filled with gas or liquid and be designed to behave incompressibly in the body once inflated and remain so until actively deflated by a the release of a valve or other means. Drainage collection ports according to one or more the embodiments can vary in shape, position and number and will be further describe herein.

With regard to the prior art, the Foley catheter allows urine to pool alongside the balloon so that the bladder is never completely empty or evacuated of urine. This pooling of urine may lead to urinary tract and kidney infections. as well as other complications. As disclosed herein, the catheters described herein are operable to drain the bladder completely and therefore may result in fewer urinary tract infections.

Another advantage attributable to one or more designs disclosed herein reduce the likelihood of incontinence. Once implanted, a Foley catheter is often treated as a foreign body that irritates the bladder resulting in periodic bladder contractions. When the bladder contracts around the Foley catheter, the undrained urine in the bladder empties around the catheter resulting in periodic incontinence episodes. In contrast devices herein are operable to fully empty the bladder, thus there is no incontinence when bladder spasm occurs.

One or more methods of catheterization disclosed herein also facilitate safer catheter insertion with less risk of injury to the patient. Traumatic insertion of Foley catheters is a common complication and can be morbid for the patient. In one common scenario, the Foley catheter balloon is inflated prematurely in the urethra after the tip of the catheter enters the bladder and urine begins to drain. In this case, the balloon is inflated in the narrow urethra (rather than within the bladder, which though elastic cannot accommodated a fully expanded balloon. Employing one or more of the catheters described herein may eliminate this complication because the balloon necessarily enters the bladder first and only upon fully passing beyond the bladder neck does urine flow into the drainage port thereby giving positive feedback that the balloon is out of the urethra whereupon it is safe to inflate the balloon. Therefore, the risk of injury to the patient's urethra during catheter insertion and placement is substantially decreased due to the definite feedback via the placement of the port and positive flow of urine.

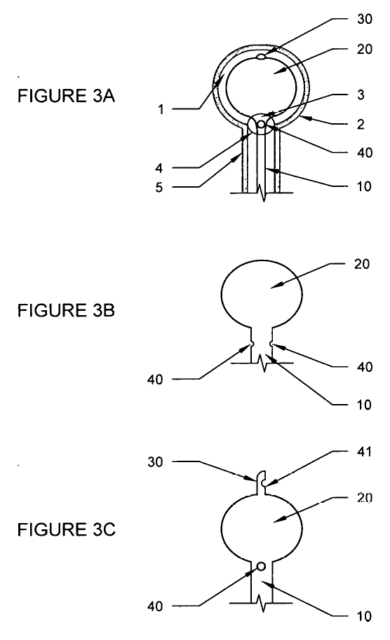

Turning to the Figures, FIG. 3A depicts an embodiment of an improved indwelling catheter 100 implanted within a bladder 1 and urethra 2. The catheter body 1 is inserted within the urethra 2. Drainage port 40 located laterally on the side of the catheter body 1 is positioned below the balloon buoyancy device or expandable anchor device 20. Various embodiments of the balloon or anchor 20 can be made of any expandable, pliable, elastomeric, polymer, or rubber like material. Other anchors can be fashioned from self-expanding, sponge-like, or hydrogel materials and self-expand upon contact with liquid or alternatively they may be released from a retaining band or retractable sleeve and thus not require inflation to function. The port 40 is shown positioned at least partially above the bladder orifice or outlet 3 which is also the beginning of the urethra 5. Alternatively, the port 40 can be implanted completely above the outlet 3, adjacent to it, or in fluid communication with it. The bladder outlet 3 is positioned on the lower inner surface or wall 2 of the bladder, thus gravity alone (and not micturition or muscular contraction) is sufficient for drainage once the catheter outlet is in place.

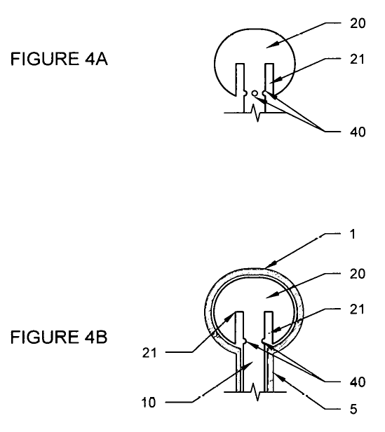

FIGS. 4A and 4B are cross-sectional side views of an embodiment of the invention 100 having a balloon with an “umbrella” shaped configuration such that the balloon presents a generally circumferential and or concave surface on its lower portion or surface 21 of the balloon, expandable anchor or retaining device 20. The at least partial concavity 21 is located proximal or adjacent to one more of the drainage ports 40. The drainage ports 40 can also be in fluid communication with at least a portion of the lower surface of the anchor 21.

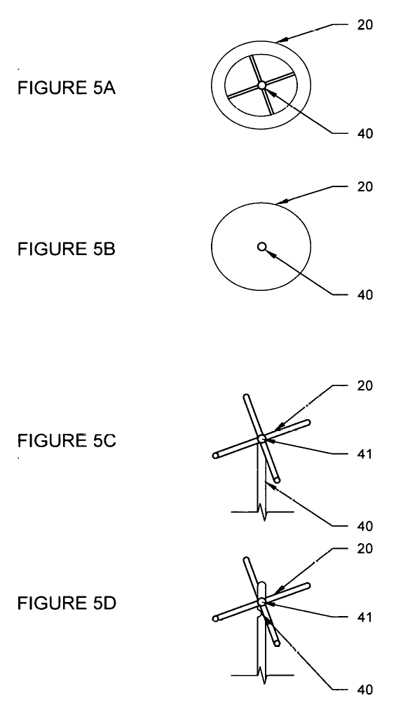

FIGS. 5 A-d show perspective views of various embodiments of the invention wherein the catheter has expandable balloon member(s) 20 embodied as a toroid, disc, radial four-legged extensions and tripod extensions.

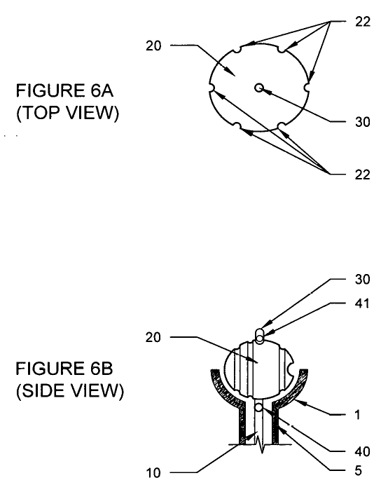

FIGS. 6A and 6B show a top view and a side view of an embodiment of the invention 100 having a balloon or expandable member 20 having exterior channels indentations, ridges, concavities on or along balloon surface 22 to allow the flow of liquid across its exterior and into the drainage ports or port 40.

Alternatively or additionally, dimples or protrusions 23 may be integrated along the exterior surface of the balloon or expandable anchor 22.

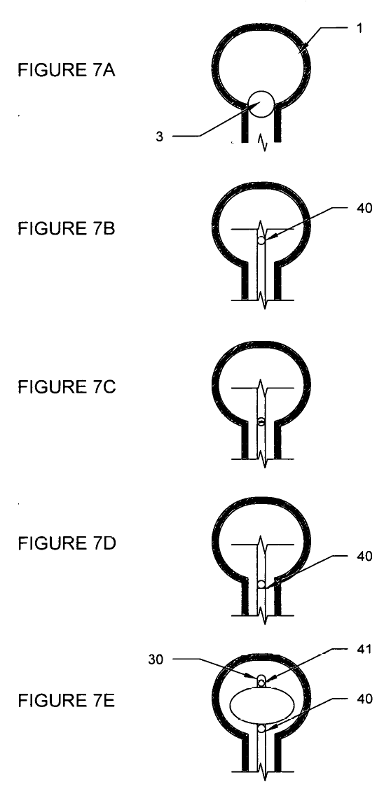

FIGS. 7A-E show side views of a bladder and urethra wherein the port 40 of a catheter device 10 is positioned at various locations relative to the intersection of the bladder and urethra or bladder outlet 3. The port 40 is shown at the junction 3 below it, above it, and halfway in between being in fluid communication with both the urethra 5 and bladder 1.

It should be understood that many of the features described herein with respect to a particular embodiment are interchangeable or usable in combination with other embodiments. It should also be understood that the device of the present invention could be designed in a number of shapes to accommodate particular bodily environments. In addition, the present invention provides for a combination of or system including any of the above-described devices, or elements that are suitably interchangeable as depicted in the figures, or those defined in the below claims. The inventions disclosed herein have been described with reference to particular embodiments, however, it is to be understood that these are merely exemplary of the principles and applications thereof. Thus, numerous embodiments and modifications may be devised without departing from the scope of the inventions disclosed herein.

1. A urinary catheter comprising:
an elongate primary tube having a proximal exit end for collecting or dispensing fluids and a distal receiving end, said primary tube having one or more drainage apertures for receiving fluids;
an inflatable anchor carried proximal to the distal end of said primary tube;
said primary tube carrying a secondary tube for passing or receiving a substance to said inflatable anchor device;
said anchor configured such that once delivered within and beyond a urethra and into a bladder and subsequently inflated therein, said anchor will resist the expulsion of the distal end of said primary tube end out of said bladder upon flow of fluid within said bladder or during micturition; and
wherein the one or more drainage apertures are laterally positioned below and proximate to said inflatable anchor device along said primary tube such that said fluids may pass into the primary tube.

2. The catheter in claim 1 wherein the one or more drainage apertures are be operable to be in partial fluid communication a bladder outlet when the catheter is inserted in the bladder and the inflatable anchor is inflated.

3. The catheter in claim 1 wherein the anchor has an upper surface and a lower surface wherein the lower surface is at least partially concave.

4. The catheter in claim 1 further comprising a second drainage aperture that is located at the distal end of the primary tube above or on the upper surface the inflatable anchor.

5. The catheter in claim 1 wherein the one or more drainage apertures are positioned radially along said primary tube.

6. The catheter in claim 1 wherein the one or more drainage apertures are oblong and positioned along a long axis of said primary tube.

7. The catheter in claim 1 wherein the one or more the drainage apertures are comprised of a circumferential mesh.

8. The catheter in claim 1 wherein the inflatable anchor is configured from a list of shapes including sphere, toroid, cylinder, and ellipsoid.

9. The catheter in claim 1 wherein the inflatable anchor further comprises radial surface indentations configured to channel fluid below said anchor towards the one or more drainage apertures.

10. The catheter in claim 1 wherein the inflatable anchor further comprises one or more surface protrusions configured to provide a gap between an outer surface of said inflatable anchor and bladder wall configured to channel fluid below said anchor towards the one or more drainage apertures.

11. An anchorable irrigation device for releasing fluid from a hollow structure within a patient comprising:
an elongate primary cannulated tube having a proximal exit end for collecting or dispensing fluids and a distal receiving end, said primary tube having one or more drainage apertures for receiving fluids;
an expandable anchor carried proximal to the distal end of said primary tube;
said anchor configured such that once delivered within the interior of said hollow structure and subsequently expanded therein, said anchor will resist the expulsion of the distal end of said primary tube end out of said vessel upon flow of fluid within said structure; and
wherein the one or more drainage apertures are laterally positioned below and proximate to said inflatable anchor device along said primary tube such that said fluids may pass into the primary tube.

12. The irrigation device in claim 11 wherein the primary tube carries a secondary tube for passing or receiving a substance to said anchor device operable to expand or contract it.

13. The irrigation device in claim 11 further comprising releasable retaining member operable to hold the anchor in a closed position.

14. A method of preventing infection within a patient's bladder comprising the steps of:
inserting a catheter having an expandable anchor at its distal end and a drainage aperture positioned below said anchor through a urethra and into and beyond a bladder outlet into the bladder,
causing or allowing said anchor to expand within the bladder beyond said bladder outlet; and
positioning at least a portion of said aperture between said anchor and a lower portion of said bladder thereby fully evacuating the bladder and preventing urine from remaining therein and causing infection.

15. The method in claim 14 further comprising the step of expanding the anchor via inflation.

16. A method of irrigating a hollow structure within a patient comprising the steps of:
inserting a catheter having an expandable anchor at its distal end and a drainage aperture positioned below said anchor through the surface and into and within the hollow structure;
expanding the anchor, and
positioning at least a portion of said aperture between said anchor and an interior surface of said hollow structure.

17. The method in claim 16 wherein the hollow structure is a bladder and further comprises the step of piecing a hole through the wall of said bladder and causing or allowing the bladder wall to seal around the catheter.

18. A method of preventing the pathological expansion of an expandable anchor attached to the distal end of an indwelling catheter within a urethra comprising the steps of:
inserting an indwelling catheter within a urethra, said catheter having a elongate primary tube having a proximal exit end for collecting or dispensing fluids and a distal receiving end, said primary tube having one or more drainage apertures for receiving fluids, an inflatable anchor carried proximal to the distal end of said primary tube, said primary tube carrying a secondary tube for passing or receiving a substance to said inflatable anchor device and wherein the one or more drainage apertures are laterally positioned below and proximate to said inflatable anchor device along said primary tube such that said fluids may pass into the primary tube;
continuing to insert the catheter into the urethra until fluid appears or emerges from the proximal collecting end of said primary tube thereby indicating that the inflatable member has been inserted beyond the urethra and completely within the bladder.
inflating the inflatable anchor and securing the drainage port within the bladder.
